SEQUENCE LISTING (25 sequences)

SEQ ID NO: 1 - DNA, 3765 bp, SARS Coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatctcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggcgatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacagttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagttaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccctttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgcgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacaca

SEQ ID NO: 2 - protein, 1255 aa, SARS Coronavirus
MFIFLLFLTLTSGSDLDRCTTFDDVQAPNYTQHTSSMRGVYYPDEIFRSDTLYLTQDLSLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGDLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTVFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - DNA, 493 bp, SARS Coronavirus
taccaccttatgtccttcccacaagcagccccgcatggtgttgtcttcctacatgtcacgtatgcgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatatta

SEQ ID NO: 4 - protein, 164 aa, SARS Coronavirus
YHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYI

SEQ ID NO: 5 - protein, 138 aa, SARS Coronavirus
RNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYI

SEQ ID NO: 6 - protein, 1132 aa, SARS Coronavirus
MFIFLLFLTLTSGSDLDRCTTFDDVQAPNYTQHTSSMRGVYYPDEIFRSDTLYLTQDLSLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGDLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTVFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKE

SEQ ID NO: 7 - protein, 1055 aa, SARS Coronavirus
MFIFLLFLTLTSGSDLDRCTTFDDVQAPNYTQHTSSMRGVYYPDEIFRSDTLYLTQDLSLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGDLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTVFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 8 - protein, 1055 aa, SARS Coronavirus
MFIFLLFLTLTSGSDLDRCTTFDDVQAPNYTQHTSSMRGVYYPDEIFRSDTLYLTQDLSLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGDLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTVFSTFKCYGVSATKLNDLCFSNVYADSFVVKGDDVRQIAPGVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 9 - protein, 1055 aa, SARS Coronavirus
MFIFLLFLTLTSGSDLDRCTTFDDVQAPNYTQHTSSMRGVYYPDEIFRSDTLYLTQDLSLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 10 - protein, 1084 aa, SARS Coronavirus
MFIFLLFLTLTSGSDLDRCTTFDDVQAPNQPIDVVRDLPSGFNTLKPIFKLPLGINITNFRAILTAFSPAQDIWGTSAAAYFVGDLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTVFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYAPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - DNA, 29 bp, Artificial Sequence
  Description of Artificial Sequence Synthetic primer
acggatccaccatgtttattttcttatta

SEQ ID NO: 12 - DNA, 27 bp, Artificial Sequence
  Description of Artificial Sequence Synthetic primer
cagtttaatgtaatgtgtatccggatg

SEQ ID NO: 13 - DNA, 28 bp, Artificial Sequence
  Description of Artificial Sequence Synthetic primer
aaggatccagatcagacactctttattt

SEQ ID NO: 14 - DNA, 26 bp, Artificial Sequence
  Description of Artificial Sequence Synthetic primer
gagacacgagatgttgagctctaaaa

SEQ ID NO: 15 - DNA, 27 bp, Artificial Sequence
  Description of Artificial Sequence Synthetic primer
caggatcctcaacctttaagtgctatg

SEQ ID NO: 16 - DNA, 29 bp, Artificial Sequence
  Description of Artificial Sequence Synthetic primer
aaagtgtttataatggactgagctctatc

SEQ ID NO: 17 - DNA, 25 bp, Artificial Sequence
  Description of Artificial Sequence Synthetic primer
ctggatccttttcgcttgatgtttc

SEQ ID NO: 18 - DNA, 26 bp, Artificial Sequence
  Description of Artificial Sequence Synthetic primer
ttacacggaaagagggagctcattat

SEQ ID NO: 19 - DNA, 27 bp, Artificial Sequence
  Description of Artificial Sequence Synthetic primer
ctggatcctcttttattgaggacttgc

SEQ ID NO: 20 - DNA, 27 bp, Artificial Sequence
  Description of Artificial Sequence Synthetic primer
tacacggtagggtcctcgagctcaagt

SEQ ID NO: 21 - DNA, 24 bp, Artificial Sequence
  Description of Artificial Sequence Synthetic primer
agggatcccaccttatgtccttcc

SEQ ID NO: 22 - DNA, 29 bp, Artificial Sequence
  Description of Artificial Sequence Synthetic primer
cgttatataatttaccggagagctccaaa

SEQ ID NO: 23 - protein, 20 aa, SARS Coronavirus
FGRDVSDFTDSVRDPKTSEI

SEQ ID NO: 24 - protein, 5 aa, Unknown Organism
  Description of Unknown Organism Peptide motif
RRARR

SEQ ID NO: 25 - protein, 5 aa, Unknown Organism
  Description of Unknown Organism Peptide motif
RRAHR

(19) United States
(12) Patent Application Publication
(10) Pub. No.: US 2007/0116716 A1
(43) Pub. Date: May 24, 2007

(54) Sars coronavirus s proteins and uses thereof

(76) Inventors: Shuo Shen, Singapore (SG); Wanjin Hong, Singapore (SG); Seng Gee Lim, Singapore (SG); Yee Joo Tan, Singapore (SG); Burtram Clinton Fielding, Western Cape (ZA); Phuay Yee Goh, Singapore (SG); Timothy Hoe Peng Tan, Singapore (SG); Jian Lin Fu, Singapore (SG)

Correspondence Address: COHEN, PONTANI, LIEBERMAN & PAVANE, 551 FIFTH AVENUE, SUITE 1210, NEW YORK, NY, 10176, US

(21) Appl. No.: 10/582,301

(22) PCT Filed: Dec. 10, 2004
(86) PCT No.: PCT/IB04/04073

§ 371 Date: Sep. 18, 2006

Related U.S. Application Data
(60) Provisional application No. 60/528,596, filed on Dec. 10, 2003.

(51) Int. Cl. A61K 39/395; A61K 39/12

(52) U.S. Cl. 424/204.1; 424/130.1

(57) ABSTRACT

The present invention relates to the use of a matured, glycosylated Spike (S) protein of SARS Coronavirus, fragments of the S protein, methods for producing the same, their use in detecting SARS infection, and their use or the use of their corresponding antibodies to treat patients suffering from SARS.

### FIELD OF THE INVENTION

The invention relates to the use of a matured, glycosylated Spike (S) protein of SARS Coronavirus, fragments of the S protein, methods for producing the same, their use in detecting SARS infection, and their use or the use of their corresponding antibodies to vaccinate or treat patients suffering from SARS.

### BACKGROUND OF THE INVENTION

In 2002-2003, a new virus that caused an atypical pneumonia, a disease termed SARS, emerged from the Southern part of China and infected people from at least 30 countries. At the end of the outbreak in July 2003, the consolidated World Health Organization number of reported patients and deaths due to SARS, was about 8500 and more than 800 respectively. This outbreak not only incurred great social and medical costs to affected countries, it also had devastating effects on regional as well as global economies.

A novel coronavirus was established to be the causative agent for SARS (ref. 7, Drosten et al., 2003; ref. 39, Ksiazek et al., 2003) and was subsequently named SARS coronavirus or SARS CoV. It's genome of 29.6 kb revealed 14 open reading frames (orfs), encoding the replicase, spike, membrane, envelop and nucleocapsid (N) which are similar to other coronaviruses, and several other unique proteins (ref. 40, Marra et al., 2003; ref. 41, Rota et al., 2003). The difficulty with the SARS Cov spike protein is that being a glycol protein it is difficult to produce enough of the protein in a humanized form that would be suitable for the production of antibodies, vaccines and other therapeutic, diagnostic and prophylactic tools. A protein in a humanized form is one that is similar to the protein form in a human body.

### SUMMARY OF THE INVENTION

One solution to this difficulty would be to find a system capable of producing a SARS CoV spike protein that is glycosylated so as to be humanized.

Another solution is to find fragments of the SARS CoV spike protein that are capable of inducing neutralizing antibodies.

In the present invention, we expressed 5 different fragments of S, covering the entire ectodomain (48 to 1192 amino acids), as GST-fusion proteins in Escherichia coli and used the purified proteins to raise antibodies in rabbits. By Western blot analysis and immunoprecipitation experiments, we showed that all the antibodies are specific and highly sensitive to their target regions. Indirect immunofluorescence performed on fixed but unpermeabilized cells showed that these antibodies can recognize linear epitopes on the ectodomain of S, which is expressed on the cell surface. When the antibodies were tested for their ability to inhibit SARS-CoV replication in Vero E6 culture, anti-S 10 antibody, which was targeted to 1029-1192 amino acids of S SEQ ID NO 4, has strong neutralizing activities, suggesting that this region of S is very important for virus entry and/or replication containing a SARS_COV neutralizing domain.

According to one aspect of the present invention there is provided a mature, glycosylated spike protein of a coronavirus.

Another aspect of the invention provides a method of producing a mature, glycosylated spike protein of a coronavirus comprising the steps:
transfecting a cell with a nucleic acid encoding a spike protein of a coronavirus or part there of; expressing the spike protein in the cell; and isolating the spike protein.

A further aspect of the invention provides a method of screening for a mature, glycosylated spike protein of a coronavirus comprising the steps:
isolating a spike protein; immunoprecipitating the isolated spike proteins with Endo-H; and detecting the remaining spike proteins that are the mature glycosylated spike protein.

Another aspect of the invention provides an antibody to a mature, glycosylated spike protein of a coronavirus or part their of.

The following statements relate to the above aspects of the invention.

In one embodiment, preferably the coronavirus is a SARS coronavirus. Preferably the coronavirus is a SARS coronavirus strain, 2774.

In another embodiment, preferably the mature glycosylated spike protein contains a transmembrane domain (TMD).

In another embodiment, preferably the mature glycosylated spike protein is a 210 KDa protein.

In another embodiment, preferably the cell is a lung cell line A549.

In another embodiment, preferably the antibody is used for immunodectection of a SARS coronaviral infection.

In another embodiment, preferably the spike protein or the antibody is used in the production of a vaccine.

According to a further aspect of the invention, the present invention provides for a peptide or protein fragment of a S protein (SEQ ID NO. 2) of the SARS coronavirus, said fragment comprising the sequence of amino acid numbers 1055 to 1192 from the S gene of the SARS coronavirus (SEQ ID NO. 5), or alternatively, the sequence of amino acid numbers 1029 to 1192 of said S gene (SEQ ID NO. 4). Preferably, the peptide or protein has the HR2 heptad region of the coronavirus S protein. The peptide or protein may be S 10 (SEQ ID NO. 4), S 11 (SEQ ID NO. 6), S 17 (SEQ ID NO. 7), S 18 (SEQ ID NO. 8), S 19 (SEQ ID NO. 9), or S 20 (SEQ ID NO. 10), as described below.

The present invention also provides for a method of producing a fragment of the S protein of coronavirus comprising the steps of:
a) transfecting a cell with a nucleic acid encoding a fragment of the S protein of coronavirus comprising the sequence of amino acid numbers 1055 to 1192 from the S gene of the SARS coronavirus (SEQ ID NO. 5), said nucleic acid in operative association with regulatory sequences capable of directing the expression thereof in the cell;
b) expressing the protein fragment in the cell; and
c) isolating the protein fragment.

The present invention additionally provides for an antibody to a peptide or protein fragment of SEQ ID NO. 2 of the SARS coronavirus, said fragment comprising SEQ ID NO. 5, preferably SEQ ID NO. 4. Preferably, the peptide or protein comprises the HR2 heptad region of the coronavirus S protein. More specifically, the present invention provides for antibodies to the peptide or protein S 10 (SEQ ID NO. 4), S 11 (SEQ ID NO. 6), S 17 (SEQ ID NO. 7), S 18 (SEQ ID NO. 8), S 19 (SEQ ID NO. 9), or S 20 (SEQ ID NO. 10).

The antibody may be used in a method of detecting a SARS coronaviral infection in a patient comprising the step of applying the antibody at least part of the cells collected from the patient. A related kit for the detection of SARS coronavirus containing the antibody is provided by the present invention.

Finally, the present invention provides for a method to treat a patient with severe acquired respiratory syndrome or prevent the onset thereof comprising administering to the patient the peptide or protein described above, or the antibody of such peptide or protein. For example, a vaccine containing comprising an effective amount of the peptide or protein, or antibody of such peptide or protein is provided by the present invention.

### BRIEF DESCRIPTION OF THE DRAWINGS

FIG. 1

Western Blot Analysis for detection of S protein. Lysates from Cos7 cells are transfected with plasmid pKT-S (Lane 1, 3, 5, 7, 9, 11). Lysates from Cos7 cells are transfected with plasmid without insert as negative control (Lane 2, 4, 6, 8, 10, 12). A BenchMark Pre-Stained Protein Ladder (Invitrogen) is used as the markers on the right. The 200 kDa and 140 kDa bands are specific S protein bands.

FIG. 2

Specificity of antibodies determined by radiolabeled immunoprecipitation. Lysates of Cos7 cells are transfected with S, S 11, S 12, S 13, S 14, S 15 and S 16 respectively (Lane 1, 2, 3, 4, 5, 6 and 7). Lysates of Cos7 cells are transfected with plasmid without insert as negative control (Lane 8). High-Range Rainbow Molecular Weight Markers (Amersham) is used as the marker on the left.

FIG. 3

Time-course of the glycosylation of S protein. Lysates of Cos7 cells transfected with PKT-S are harvested at Oh, 0.5 h, 1 h, 2 h, 4 h and 6 h respectively (Lane 1, 2, 3, 4, 5, 6 and 7). Lysates of Cos7 cells transfected with plasmid without insert are harvested at 6 h as negative control (Lane 8). Lysates of Cos7 cells are transfected with pKT-S and treated EndoH (6 h post-transfection). Lysates of Cos7 cells transfected with pKT-S and harvested at 6 h post-transfection as negative control. Rabbit anti-S 10 was used for the detection of S protein.

FIG. 4

S protein expressed on the surface of transiently transfected Cos7 cells. S protein expressed on the surface of Cos7 cells, were detected by Rb-S 1, Rb-S 2, Rb-S 3, Rb-S 9 and Rb-S 10 respectively, as indicated by the green fluorescence. Positions of the cells were taken using white light. No fluorescence were detected in control experiments.

FIG. 5

Mapping of sequence harboring the cleavage site of S1/S2. The full-length S (lanes 1 and 6), internal-deletion mutants S 32 (lanes 2 and 7), S 33 (lanes 3 and 8) and S 34 (lanes 4 and 9) lacking residues 531-550, 551-570 and 571-590, respectively, were expressed in Cos-7 cells. Western blot analysis was performed using rabbit-S 10 (left panel) and horse-SARS (right panel). Proteins were separated on 10% PAGE gels. Molecular masses of specific proteins are indicated on the right and masses of markers are indicated on the left in kilodalton. The same gels were stripped and reprobed with mouse anti-actin as loading controls.

FIG. 6

Treatment of the S-derived proteins with N-glycosidase F and Endoglycosidase H. The full-length S was expressed in Cos-7 cells. Cells were resuspended in lysis buffer. The samples were treated (+) or mock-treated (−) with (a) N-glycosidase F (N-gly-F) and (b) Endoglycosidase H (Endo-H). Proteins were separated on 10% PAGE gels and Western Blot was performed with rabbit-S 1. Lysates from mock-transfected cells were used as negative controls (lanes 3, 4, 7 and 8). Molecular masses of specific proteins are indicated on the right and masses of markers are indicated on the left in kilodalton.

### DETAILED DESCRIPTION OF THE PRESENTLY PREFERRED EMBODIMENTS

The S protein of coronavirus is an important determinant of tissue tropism, as it binds to cellular receptors on the host cell and it is also crucial for virus and cellular membrane fusion. For SARS-CoV, it appears that humoral responses against S alone are sufficient to protect against SARS-CoV infection (14).

The S protein of Sars coronavirus strain, 2774, was expressed in monkey kidney cells Vero E6 and Cos-7, and in human kidney 293T, lung cells A549 and MRC-5 in a vaccinia-T7 expression system. The S protein was detected by immunoprecipitation (IP), western blot (WB), immunofluorescence (IF), when poly- and mono-clonal antibodies against S, raised in rabbits, horse and mice, were used. These antibodies recognize different regions, covering the whole ectodomain of S. We found that, in a pulse-chase experiment, a 200 kD, core-glycosylated form of S was processed into a 210 kD, fully glycosylated, endo-H resistant form. Furthermore, when a set of C-terminally-truncated and internal-deletion constructs were expressed, it was found that the S polypeptides without transmembrane domain (TMD) were endo-H sensitive, not transported to the trans-Golgi and not expressed on the cell surface, in contrast to those with TMD. We also found that two host proteins (62 kD and 64 kD, respectively), in every cell lines tested, were co-immunoprecipitated with S by antibodies raised in animals and antibodies from recovered patients. Amino acid residues 31 to 200 of S were identified to be the domain that interacted with these two proteins, as the deletion of this region completely abolished the specific binding. These two proteins are glycoproteins as they are sensitive to endo-H treatment and they interact with the S protein of Sars coronavirus but not with that of IBV, a member of the group 3 coronaviruses of birds. Interestingly, the S proteins were cleaved into at least two pieces in transfected cell detected in WB experiments, although no conventional cleavage sites were predicted by sequence comparison. In virus-infected cells, similar cleavage products were also observed. The S protein could be detected in the supernatant of infected cells, roughly half of which were cleaved. We found that the most efficient expression and maturation occurred in transfected Cos-7 and A549 cells in parallel transfection experiments. The processing is more complete in A549 cells than in Cos-7 cells, as the majority of the S proteins are the matured, fully-glycosylated 210 kD form, which co-migrates with the native form of the S protein in the supernatant of virus infected cells.

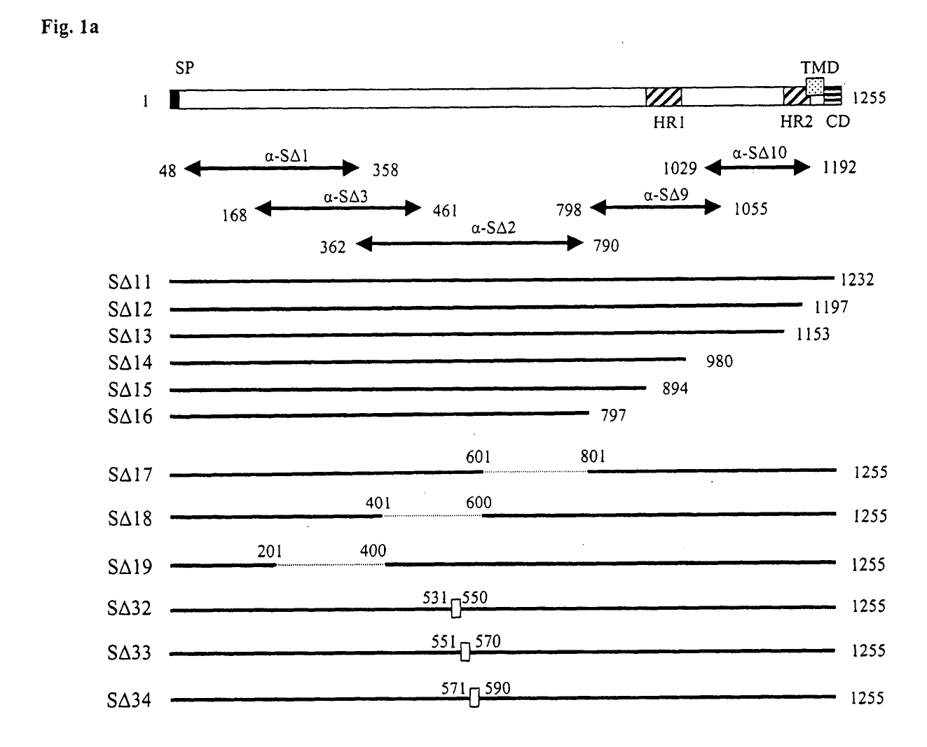

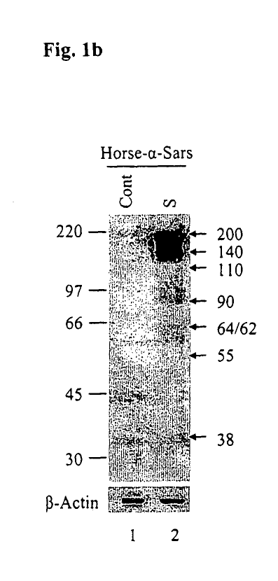

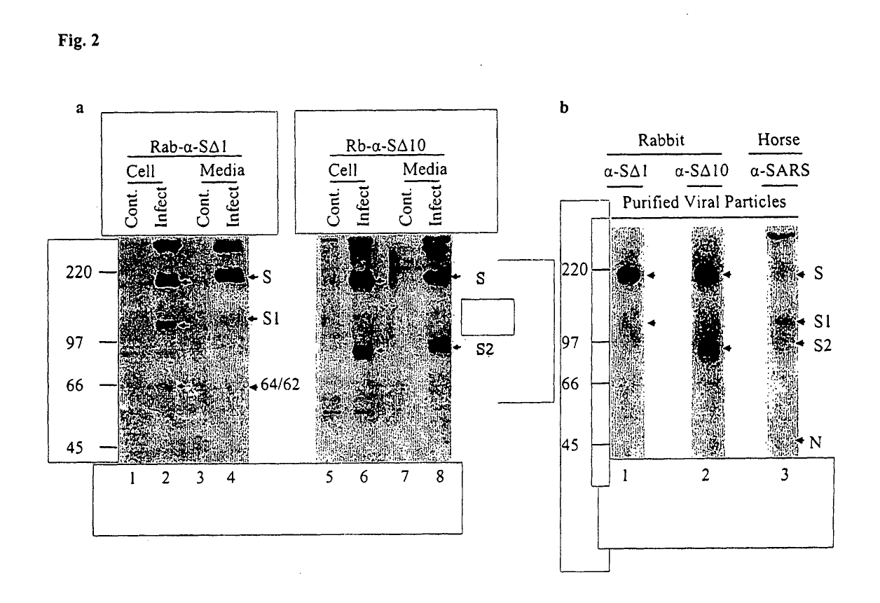

In the present invention, we have also obtained fragments of the S protein and have raised rabbit polyclonal antibodies against 5 bacterially expressed S fragments, covering the entire ectodomain (48 to 1192 amino acids). As these proteins were expressed in Escherichia coli, the antibodies will not be able to recognize some post-translational modifications on S such as glycosylation sites. Our analysis is also likely to be limited to linear epitopes since the proteins were extracted from SDS denaturing gels and eluted in the presence of SDS although we cannot rule out that the protein can maintain some degree of folding. Interestingly, we found that the antibodies were not only able to recognize denatured S proteins (expressed in mammalian cells) in Western blot analysis, but could also be used to immunoprecipitate native S proteins from cell lysates (FIGS. 1 and 2). The results indicate that these antibodies are binding to linear and exposed epitopes present in the folded S and/or to flexible regions, like turns, that are found in the structure of S. Importantly, immunoprecipitation experiments showed that these antibodies could bind to both the unglycosylated (140 kDa) and core-glycosylated (200 kDa) full-length S, as well as the EndoH resistance fully-glycosylated S (210 kDa). This is also supported by indirect immunofluorescence experiments which showed that these antibodies could recognize S protein expressed on the cell surface. It is known that the core-glycosylated S protein has to be transported to the Golgi apparatus before it undergoes maturation, resulting in an increase in apparent molecular weight from 200 kDa to 210 kDa, and is then transported to the cell surface (11, 15). It is also known that the 210 kDa fully-glycoslyated form of S is later incorporated into virions and secreted into the medium of SARS-CoV infected Vero E6 culture.

Neutralization assays showed that antibodies raised against GST-S 10 were capable of neutralizing SARS-CoV replication in Vero E6 cells at a titre of up to 1:364 at 200 TCID₅₀, which is comparable to the level obtained for convalescent patients. Analysis of sera taken after accumulative immunizations displayed a steady increase in neutralizing titer, indicating that the immunized rabbits were showing a specific immune response to GST-S 10. None of the other antibodies appear to be capable of inducing neutralizing antibodies, which could indicate that there was an absence of neutralizing epitopes in 48-1055 amino acid (aa) of S protein. However, this phenomenon is most unlikely as a human antibody against 261-672 aa has been shown to have neutralizing properties (see below for discussion on receptor binding domain). The more probable explanation is that neutralizing epitopes in this region are heavily glycoslyated and/or contain rigid tertiary structures. Therefore, our results showed that the 48-1055 aa of S protein is less suitable for use in the development of vaccines that are peptide-based or based on proteins expressed in non-mammalian systems that are not able to mimic the glycoslyation process in human cells. Since S 9 is from 798-1055 aa and S 10 is from 1029-1192 aa, it is clear that the region 1055 to 1192 aa of S protein SEQ ID NO. 5 contains linear neutralizing epitope(s) that would be suitable for subunit vaccine development.

The first step in coronavirus infection is the attachment of virions to host cells and in the case of SARS-CoV, ACE-2 has been identified as the cellular receptor that binds to the SARS-CoV S protein (9). A domain in the N-terminal of S protein, approximately 300 to 510 amino acid (aa), is the receptor binding domain (16). Subsequently, the fusion of the lipid bilayer of the viral envelope with the host cell membrane occurs and this process is also mediated by the S protein (4). The coronavirus S protein is a class I virus fusion protein and contains two heptad repeat regions (HR1 and HR2) are found in S2 domain or C-terminal domain. These domains are postulated to play an crucial role in defining the oligomeric structure of S and hence mediate the fusion between viral and cellular membranes (4). For the SARS-CoV, HR2 is located close to the transmembrane anchor (1148-1193 aa) and HR1 is ˜140 aa upstream of it (900-1005 aa) (14). Interestingly, S 10 (1029-1192 aa) encompasses the HR2 region.

Biochemical studies have shown that peptides corresponding to the HR1 and HR2 of SARS-CoV S protein can associate into an anti-parallel six-helix bundle with structural features typical of the other known class I fusion proteins, suggesting that the membrane fusion and cell entry mechanisms exploited by SARS-CoV is similar to that for other coronaviruses like MHV (1). In the full-length S protein, the HR1-HR2 structure brings the fusion peptide, predicted to be near the N-terminal of HR1, into close proximity to the transmembrane domain and this facilitates the fusion between viral and cellular membranes, allowing the virus to enter the cell. It would be very probable that by binding to the HR2 domain with high affinity, anti-S 10 antibodies could block the interaction between HR1 and HR2 and hence prevent the SARS-CoV fusion with the host cells.

As bacterially expressed proteins would be easier and cost-effective to produce on a large scale, the S 10 fragment (1029-1192 aa) SEQ ID NO. 4 identified in this study may be an ideal vaccine candidate for SARS-CoV. In future studies, it will be critical to determine if anti-S 10 antibodies can protect against SARS-CoV infection in animal models and to delineate the precise contribution of this S region to membrane fusion. For the latter, we are currently mapping monoclonal antibodies obtained from mice that have been immunized with GST-S 10 proteins and determining their binding and neutralizing properties. Lastly, recombinant S 10 protein and anti-S 10 antibodies can potentially be developed for anti-viral treatments.

Coronaviruses are positive-strand RNA viruses and the virion consists of a nucleocapsid core surrounded by an envelope containing three membrane proteins, spike (S), membrane (M) and envelope (E), which are common to all members of the genus (for review, see 8, 13). The S protein, which forms morphologically characteristic projections on the virion surface, mediates binding to host receptors and membrane fusion. The M and E proteins are important for viral assembly while N is important for viral RNA packaging.

The S protein of coronavirus is responsible for inducing host immune responses and virus neutralization by antibodies (6, 14). For SARS-CoV, it may be that prior infection provides protective immunity in a mouse model and the passive transfer of neutralizing antibodies to naïve mice also protect them from infection. This would involve, no enhancement of SARS-Cov infection in mice upon re-infection or after the administration of immune serum, unlike the case for feline infectious peritonitis virus (10), and therefore, it would be safe to have a vaccination against SARS-CoV. A DNA vaccine encoding the S protein alone may induce T cell and neutralizing antibody responses and protect mice from SARS-CoV infection, suggesting the S protein is indeed the primary target for viral neutralization in SARS-CoV infection. This finding was also confirmed by an independent study that uses surrogate/carrier virus to express S in primates (5). From these studies, it appears that humoral response against S alone is sufficient to protect against SARS-CoV infection.

Although inactivated whole SARS-CoV may show promising results for vaccination, it is important to identify epitope(s) in S capable of inducing neutralizing antibodies as epitope-based vaccines would avoid any possibility of reversion to virulence and would be safer and cheaper to produce. In this study, we identified neutralizing epitopes in the S protein of SARS-CoV, to be used for the development of vaccines or therapeutic agents against SARS-CoV infection. We expressed different regions of S as GST-fusion proteins and used them to raise antibodies in rabbit. These polyclonal antibodies were then tested for their specificities towards the S protein in Western blot analysis, immunoprecipitation and immunofluorescence analyses as well as for their capacities to inhibit SARS-CoV replication in Vero E6 culture.

For members of the coronavirus family, previous studies have shown that, in most cases, the S protein is cleaved into two subunits, the N-terminal S1 and C-terminal S2, probably by cellular furin-like proteases. The proteolytic cleavage might enhance membrane fusion and infectivity but may not be a prerequisite (ref. 20-23). It seems that the relationship between the cleavage and membrane fusion (or infectivity) is dependent on individual strains and cells used (24). Perhaps this reflects the multiple determinants of the fusion activity of S and difference in expression level of enzymes in the cells tested. The proteolytic cleavage of the S protein of SARS CoV has not been found to date. Therefore, it would be interesting to investigate whether the S protein of SARS CoV was cleaved into S1 and S2. If it is also cleaved, then the effect on the fusion and infectivity could be studied for this newly-emerged coronavirus. We found that the S protein of SARS CoV was cleaved into an N-terminal S1 of 110-kDa and a C-terminal S2 of 90-kDa as they were detected in the media of infected Vero E6 cells and in purified virions by using antibodies specific to the N- and C-termini of S. As the full-length S protein of 200-kDa was also detected in virions, we concluded that the S protein of SARS CoV was partially cleaved. The relative abundance of cleavage products could not be accurately estimated by directly scanning the blots with a densitometer because the affinities of antibodies to S1 and S2 are different. These antibodies were raised with purified virions and E-coli-expressed S fragments, respectively.

The fact that a typical S1/S2 cleavage signal for other coronaviruses is absent in the S sequence of SARS CoV makes the finding more interesting. Previous studies suggest that RRARR or RRAHR motif is located immediately upstream of the cleavage site of the coronavirus S protein. Point mutation of some of the multiple basic residues might impair or abolish the cleavage (ref. 23, 25). Presumably, this motif is required for furin-like proteases of mammalian cells, which has been shown to be responsible for cleavage of other viral fusion proteins (ref. 26). We decided to develop an expression system of the single S protein in a mammalian cell line, and then by using mutants to determine the alternative cleavage motif for S of SARS CoV. A 90-kDa protein with similar size as the S2 identified in infected cells was detected in Cos-7 cells transfected with the S gene SEQ ID 1. The system makes it possible to determine the cleavage signal by using internal-deletion mutants of S. We found that when residues from 551 to 570 were removed, the 90-kDa protein was not detected. The sequence of the 20 residues deleted in the mutant was FGRDVSDFTDSVRDPKTSEI. The 90-kDa protein was the cleavage protein S2 in cells transfected with the S gene. The 20 amino acid residues did not contain an alternative translation-initiation codon, so the 90-kDa protein could not be due to internal-initiation of translation. The 90-kDa protein was only detectable by antibodies against the C-terminus but not by those against the N-terminus, thus it could not be a product derived from premature-termination of translation. Interestingly, these 20 residues did not contain the motif of multiple or paired basic residues required by furin-like proteases. Therefore, the S protein of SARS CoV might not be cleaved by the same enzymes used for other coronaviruses but by different cellular endoproteases. The result is consistent with observations by others that co-expression of S with mouse furin in insect cells did not cause the S cleavage of SARS CoV. Also, no sequence motif Gln(Glu)-X-Arg (where X is Gly, Ser, or Thr) for blood clotting Factor Xa-like (or trypsin-like) protease existed, which was the consensus cleavage motif of fusion proteins of influenza A and Sendai viruses. Proteolytical cleavage activated the fusion proteins of these viruses. Based on earlier studies of the S protein of mouse hepatitis virus, the S protein is N-linked-glycosylated and trimerized in the endoplasmic reticulum (ref. 10). The high mannose side chains are trimmed and further modified during transport to the Golgi apparatus. The cleavage occurs in the Golgi apparatus or the post-Golgi. The S protein of SARS CoV contains 1255 residues and carries 23 potential N-linked glycosylation sites. If the cleavage occurs at residues 551-570, the S1 and S2 would harbor 12 and 11 sites, respectively. As the S1 was about 20-kDa larger than the S2, it was reasonable to assume that most, if not all, potential sites in S1 were used for glycosylation but not all of those in S2. In this study, we also observed that the sizes of the cleaved S1 and S2 detected in cell lysates were slightly smaller than those detected in supernatants and purified virions. This might indicate that (1) further modification occurred after cleavage of S and perhaps before assembly of virions, and (2) the S protein might be cleaved intracellularly by host cell proteases.

In Cos-7 cells transfected with the single S gene, small N-terminal products 55- and 38-kDa in size were detected but not the 110-kDa S1, unlike in Vero E6 cells infected with SARS Cov. These might be the cleaved/degraded products of S1 or premature-termination products. Interestingly, the other two proteins of 64/62-kDa were identified in both transfected and infected cells, suggesting that the production of these two N-terminal proteins is not artificial in transfected cells.

The relationship between the cleavage of S and its functions (membrane fusion, infectivity and pathogenesis) remains conflicting. Questions on the S processing still need to be addressed. For example, why is the cleavage of S evolutionally conserved in many coronaviruses if it is not linked to the functions of S? Recent report indicated that trypsin activation of pseudovirions HIV(S) with incorporated S protein of SARS CoV was required for cell-cell membrane, although it did not enhance infectivity (32).

Materials and Methods

Materials. The Cos7 and Vero E6 cells used in this study were purchased from American Type Culture Collection (Manassas, Va., USA). Cos7 cells were cultured at 37° C. in 5% CO₂ incubator in Dulbecco modified Eagle medium containing 1 g of glucose/liter, 2 mM L-glutamine, 1.5 g of sodium bicarbonate/liter, 0.1 mM nonessential amino acids, 0.1 mg of streptomycin/ml, 100 U of penicillin, and 5% fetal bovine serum (HyClone, Utah, USA). Vero E6 cells were cultured at 37° C. in 5% CO₂ incubator in Medium 199 containing 2 mM L-glutamine and L-amino acid (HyClone, Utah, USA).

The Singapore strain SARS-CoV 2003VA2774 (“2774”) of Sars coronavirus was isolated in Tan Tock Seng Hospital and adapted to grow in Vero E6 cells in laboratory of Environmental Health Institute (EHI), Singapore. Passage 3 in Vero E6 cells were used for direct RNA extraction, reverse transcription and polymerase chain reaction (RT-PCR) and sequence analysis. Recombinant vaccinia/T7 virus (VT3) was grown and titrated on Vero cells, which is a subclone for growth of avian infectious bronchitis coronavirus, IBV (ref. 54, Shen and Liu, 2003).

Cloning of DNA constructs. (a) For expression in E. coli. The Singapore isolate 2774 containing the full-length S (1-1255 amino acids (aa)) was used to perform amplification and RT-PCR, and the cDNA from the RT-PCR was used as the template for cloning of S constructs. Five constructs S 1, S 2, S 3, S 9 and S 10 (Table 1) were obtained by PCR with the primers listed in Table 2. All primers used were purchased from Research Biolabs or Proligo Pte Ltd (Singapore). The PCR products were digested by BamHI/XhoI and ligated into BamHI/XhoI-cut pGEX4T1 vector (Amersham Pharmacia Biotech, Uppsala, Sweden) to obtain plasmids pGEX-S 1, pGEX-S 2, pGEX-S 3, pGEX-S 9 and pGEX-S 10 for the expression of glutathione S-transferase (GST) fusion proteins. (b) For expression in mammalian cells. The construction of pKT-S, containing the full-length S, which can be used with the T7 vaccinia virus system to express S efficiently in mammalian cells. Plasmids pKT-S 11, pKT-S 12, pKT-S 13, pKT-S 14, pKT-S 15 and pKT-S 16 (FIG. 1) were cloned as follows.

TABLE 1
Plasmids used in this study

| | | Amino | Total |
|---|---|---|---|
| Plasmids | Nucleotide sequence | acid position | no. of amino acids |
| pKT-S | 1-3765 | 1-1255 | 1255 |
| pGex-S 1 | 144-1074 | 48-358 | 310 |
| pGex-S 2 | 1086-2370 | 362-790 | 428 |
| pGex-S 3 | 504-1383 | 168-461 | 293 |
| pGex-S 9 | 2394-3165 | 798-1055 | 257 |
| pGex-S 10 | 3087-3576 | 1029-1192 | 163 |

TABLE 2
Oligo-nucleotide Primers used in this study

| | | |
|---|---|---|
| Primers | Sequence | Application |

| | | |
|---|---|---|
| SS03-56 | 5′-ACGGATCCACCATGTTTATTTTCTTA | Cloning |
| | TTA-3′ | full-length S |
| SS03-57 | 3′-GTAGGCCTATGTGTAATGTAATTTGA | Cloning |
| | C-5′ | full-length S |
| SS03-62 | 5′-AAGGATCCAGATCAGACACTCTTTAT | Cloning S 1 |
| | TT-3′ | |
| SS03-63 | 3′-AAAATCTCGAGTTGTAGAGCACAGA | Cloning S 1 |
| | G-5′ | |
| SS03-60 | 5′-CAGGATCCTCAACCTTAAGTGCTAT | Cloning S 2 |
| | G-3′ | |
| SS03-61 | 3′-CTATCTCGAGTCAGGTAATATTTGTG | Cloning S 2 |
| | AAA-5′ | |
| SS03-68 | 5′-CTGGATCCTTTTCGCTTGATGTTTC- | Cloning S 3 |
| | 3′ | |
| SS03-69 | 3′-TATTACTCGAGGGAGAAAGGCACAT | Cloning S 3 |
| | T-5′ | |
| SS03-01 | 5′-CTGGATCCTCTTTTATTGAGGACTTG | Cloning S 9 |
| | C-3′ | |
| SS03-02 | 3′-TGAACTCGAGCTCCTGGGATGGCACA | Cloning S 9 |
| | T-5′ | |
| SS03-03 | 5′-AGGGATCCCACCTTATGTCCTTCC- | Cloning S 10 |
| | 3′ | |
| SS03-04 | 3′-AAACCTCGAGAGGCCATTTAATATAT | Cloning S 10 |
| | TGC-5′ | |

Construction of Plasmids-Specific forward and reverse primers were designed to amplify the S gene of Singapore strain 2774. The PCR products were digested with BamHI and StuI and ligated into BamHI/EcoRV-cut pKT0, resulting in plasmid PKT-S under the control of a T7 promoter. Specific primers were designed to amplify strain 2774 sequence from nucleotide positions 21476-25171, -25066, -24934, -24415, -24157, and -23866, respectively. The six RT-PCR products were digested with BamHI and ligated to BamHI/EcoRV-cut pKT0 under the control of a T7 promoter, giving rise to plasmids pKT-S 11, pKT-S 12, pKT-S 13, pKT-S 14, pKT-S 15, pKT-S 16 and PKT-S 22, respectively. Sizes of proteins encoded by these S constructs are shown in FIG. 1a. Two-round PCR were performed using specific primers to produce S fragments with internal-deletions. The PCR fragments were cloned into pKT0, giving rise to plasmids pKT-S 17, PKT-S 18, pKT-S 19, pKT-S 32, pKT-S 33, and pKT-S 34. These mutants encode the S proteins with deletions of 200 or 20 amino acid residues at positions indicated in FIG. 1a.

Analysis of the S Protein in Infected Vero E6 Cells. Confluent cells were infected with strain 2774 at a multiplicity of infection (m.o.i) of 1 and were incubated at 37° C. for 12 to 15 h. Cell debris in the medium was removed by low speed centrifugation. Cells were washed with PBS and were resuspended in PBS. One volume of 5× standard protein sample buffer was added to four volumes of cell suspension or cultured medium. The samples were heated at 100° C. for 5 minutes and were kept at minus 20° C. before Western blot analysis. For virus purification, -propiolactone was added to infected cell culture to a final concentration of 0.05% to inactivate infectivity. The inactivation was examined by titration of treated samples in Vero E6 cells. The viruses were harvested by freezing/thawing 3 times and cell debris was removed by centrifugation at 5,000 rpm for 10 minutes. Ultrafiltration was performed to concentrate viruses (300,000 NMWL, Millipore). The concentrated sample was applied to Sepharose 4B fast flow column (Pharmacia) following manufacturer's instruction. The eluted fractions were examined by transmission electron microscope. The fraction containing virus particles was used for analysis of the S protein by Western blot.

Analysis of the S Protein in Transfected Cos-7 Cells-Fifty percent of confluent monolayer of cells in 60 mm Petri dish was infected at a m.o.i. of 1 with recombinant vaccinia virus vTF7-3 expressing bacteriophage T7 RNA polymerase. After 1 hour adsorption, cells were transfected with 2 to 5 μg of plasmid using Effectene Reagents (QIAGEN) according to manufacturer's instruction. Transfected cells were incubated overnight at 37° C. and the cell lysate was prepared by resuspending cell pellet in 1× protein sample buffer for Western blot analysis.

Purification of the recombinant S proteins expressed in E. coli. Plasmids pGEX-S 1, pGEX-S 2, pGEX-S 3, pGEX-S 9 and pGEX-S 10 were separately transformed into BL21 (DE3) cells. A single colony from each plate was grown at 37° C. overnight in LB-agar plate containing ampicillin (100 g/ml). Five milliliters of the resulting cultures was inoculated into 2 liters of LB medium containing ampicillin (100 g/ml) and was incubated in a shaker at 37° C. until OD₆₀₀ reached 0.6. Expression of proteins was induced using 1 mM IPTG. Cells were harvested 2 h after induction by centrifugation at 5,000 g for 10 min at 4° C. The cell pellets obtained were resuspended in PBS-1 mM PMSF-20 g/ml DNase I and lysed by two passages through a French Press. Lysates were centrifuged at 22,000 g for 30 min. The insoluble proteins in pellet was washed 3 times and resuspended in PBS containing 1% Triton X-100. Proteins were separated in 10% PAGE-SDS gels. Gel strips containing GST-fusion protein were cut and the proteins were eluted using Mini Trans-Blot cell (BIORAD, Hercules, Calif., USA)). The resulting fusion proteins were detected in Western Blot using mouse anti-GST antibodies (Santa Cruz Biotechnology, Santa Cruz, Calif., USA) and their concentrations were estimated by comparison with BSA standards in Coomassie Brilliant Blue R-250-stained SDS-PAGE gel.

Generation of antibodies against the various S constructs. One milligram of each of the different antigens were mixed with an equal volume of Complete Freud's adjuvant (Sigma, St. Louis, Mo., USA) and used for the immunization of New Zealand white rabbits. 2 rabbits were used to raise antibodies against each respective antigen. Two weeks after the initial immunization, the rabbits were given booster injections at three-week intervals. Incomplete Freud's adjuvant (Sigma) was used for subsequent booster injections. 10 ml of blood were harvested from the rabbits each time after the 4^{th}, 6^{th}, 8^{th}, 12^{th}, 14^{th and} 16^{th} injections. All procedures on the use of laboratory animal are done in accordance with the regulations and guidelines of Animal Research Ethics Committee (AREC).

Expression of recombinant S fragments in mammalian cells. Cos7 cells were used as the mammalian expression system for Western Blot analysis, immunoprecipitation and immunofluorescence. Monolayer of Cos7 cells, grown in a 60 mm dish were subjected to T7 vaccinia virus infection at a multiplicity of infection (m.o.i) of 1, for an hour. Transient transfection of cells with PKT-S, pKT-S 11, pKT-S 12, pKT-S 13, pKT-S 14, pKT-S 15 and pKT-S 16 plasmid were carried out using Effectene transfection reagents (Qiagen, Valencia, Calif., USA) according to manufacturer's protocol.

For control experiments, cells were infected with T7 vaccinia virus and mock transfected with empty vector, pKT0.

Endoglycosidase H and N-glycosidase F Treatments of the S Proteins—Cells were infected with recombinant vaccinia-T7 viruses and transfected with plasmid pKT-S or mock-transfected with empty plasmid as described above. The cells were washed with PBS and resuspended in lysis buffers recommended by manufacturer (Roche). Cell debris was removed by centrifugation at 12,000 rpm. Ten μl of the supernatant were mixed with 10 μl of buffers recommended by manufacturer (Roche) with or without Endo-H (0.5 units/20 μl, Roche) and incubated for 2 to 3 h at 37° C. Lysis buffers used contain protease inhibitors Complete® cocktail tablets at concentration recommended by manufacturer (Roche) and 0.2 mM PMSF.

Western Blot Analysis. Cell lysates were prepared in 1×SDS loading buffer under reducing conditions (1% SDS with 0.2 M DTT), resolved in 10% PAGE gel and transferred to a nitrocellulose membrane. The blots were blocked in 5% non-fat milk in PBS with 0.05% Tween-20 and probed with rabbit anti-sera (1:20,000 dilutions), raised against the various S fusion proteins, at 4° C. overnight. The membranes were incubated with goat anti-rabbit horseradish peroxidase (HRP)-conjugated secondary antibodies at a dilution of 1:2000 for 1 h at room temperature and developed with enhanced chemiluminescence reagent (Pierce, Rockford, Ill., USA).

Radiolabeled immunoprecipitation. Cells were infected with T7 vaccinia virus and transfected with the PKT-S or plasmids expressing C-terminal deletion mutants of S, pKT-S 11, pKT-S 12, pKT-S 13, pKT-S 14, pKT-S 15 or pKT-S 16 as described above. Cells mock-transfected with PKT0 were set up as a control. The cells were starved for 30 min before labeling with ³⁵S-met for 1½ h and chased for 2 h. For the time-course experiment, the chase period before harvesting the cells was 0 h, ½ h, 1 h, 2 h, 4 h and 6 h respectively. Cells were lysed using lysis buffer containing 50 mM Tris, 1 mM PMSF, 1% NP40 (pH 7.4), and centrifuged at 16,000 g for 10 min. 300 l of the supernatant are incubated for ½ h with 5 l of rabbit anti-GST-S 1, 2, 3, 9 or 10 followed by 1 h incubation with Protein-A sepharose beads (Roche Diagnostics). The beads were washed 3 times with lysis buffer. 20 l of 1×SDS loading buffer (containing 0.2 M DTT) were added to the beads and boiled for 10 min at 100° C. Samples were treated with Endoglycosidase H (EndoH) enzyme (Roche Diagnostics) and incubated at 37° C. for 2 h in the EndoH experiment. Samples were separated in a 7.5% SDS-PAGE gel and developed with autoradiography.

Indirect Immunofluorescence. Cos7 cells grown on Permanox slide (Nalge Nunc International, Naperville, Ill., USA.) were infected and transfected as described above. After 8 h post-transfection, cells were fixed with 4% paraformaldehyde for 10 min at room temperature and blocked with PBS plus 1% bovine serum albumin (BSA) for 30 min and then incubated with the primary antibody (1:200) for 1.5 h, washed, and then incubated with fluorescein isothiocyanate (FITC)-conjugated secondary antibody (1:200; Santa Cruz) for 1 h. All incubations and washes were performed at room temperature. Slides were mounted with Fluorescence Mounting Medium (DakoCytomation) and analyzed on a AxioVision Fluorescence Light Microscope (Carl Zeiss, Germany).

Neutralization Assay. To determine neutralizing antibodies in the rabbits' serum, we performed an assay with serial dilution of sera using a 96-well plate. 2×10⁴ Vero E6 cells were grown in 200 l of Medium 199 in each well of the 96 well plates and incubated at 37° C. Serial dilution of rabbit sera with medium in the ratio 1:10 to 1:1280 were prepared. 0.1 ml of the diluted rabbit anti-sera were mixed with 0.1 ml of SARS-CoV at 200 TCID₅₀ for an hour at room temperature before adding into the respective wells. The 96-well plate was incubated in a CO₂ incubator for 3-5 days to observe the cytopathic effect (CPE). Percentage of cells with CPE was determined by taking 10 l of resuspended cells from each well and counted under a microscope. The formulae to calculate the neutralizing titer is Log(50% Neutralizing Titer of Tested Sera)=Log(Sera Dilution Higher than 50% of CPE %)−Log(Coefficient of Dilution)×(CPE % Higher than 50%−CPE % at 50%)/(CPE % Higher than 50%−CPE % Lower than 50%). The inverse logarithm for the calculation above is determined as 50% neutralizing titer of the tested serum. All experiments are carried out in duplicates.

Results

Processing of S in A Vaccinia-T7 Expression System—To analyze expression and processing of S in transfected cells, the S gene was cloned into a vector (pKT0) under the control of a T7 promotor. Cos-7 cells were infected with vaccinia-T7 recombinant viruses and were subsequently transfected with plasmid containing the S gene. The S protein expression profile was analyzed by Western blot using horse-SARS antibodies, generated with killed and purified viral particles. As shown in FIG. 1b, the 200-, 140-, 110-, 90-64/62-, 55- and 38-kDa proteins were detected (lane 2). These species were S-specific and were likely due to the expression and processing of S as no such proteins were detected in negative control cells (lane 1). The 200- and 140-kDa proteins corresponded to the full-length S protein (glycosylated and unglycosylated), and other smaller products were probably the cleaved proteins and/or premature translation products. The results suggested that the S protein of SARS CoV might be cleaved in this in vitro expression system, which could be used for analysis of processing of SARS CoV S protein.

Detection of the Cleavage Products in SARS-CoV-Infected Cells—To investigate if the S protein is also processed in virus-infected Vero E6 cells, western blot was performed to detect the S-derived products in both the cell lysate and the media of virus-infected culture. If the S protein was cleaved into the N-terminal S1 and C-terminal S2, they should be detected in the cell lysate; and if the two cleavage proteins were assembled into virions, they should be detected in the media of infected cells. For this purpose, two rabbit anti-S antibodies were used, which were raised against the N-terminal and the C-terminal regions, respectively. Rabbit-S 1 recognized a region from amino acid resides 48 to 358 and rabbit-S 10 recognized a region from 1029 to 1192 (see FIG. 1a). As shown in FIG. 2a, when rabbit-S 1 was used, two specific proteins of 200- and 110-kDa were detected both in the cell lysate (lane 2) and in the media (lane 4). When rabbit-S 10 was used, two proteins of 200- and 90-kDa were detected both in the cell lysate (lane 6) and in the media (lane 8). The results suggested that (1) the 110- and the 90-kDa species might represent the N- and C-terminal cleavage products S1 and S2, respectively, of S in virus-infected cells and (2) the S protein was partially but not completely cleaved for SARS CoV. It was observed that the 200-, 110- and 90-kDa proteins in the media were slightly larger than their counterparts in cell lysates. It is likely that these products may have undergone further modification after cleavage and before assembly into virion.

To confirm the above observations, purified virus particles were used in Western blot using the same antibodies. As shown in FIG. 2b, the S1 subunit was detected using rabbit-S 1 (lane 1) and the S2 subunit was detected using rabbit-S 10 (lane 2). Both the S1 and S2 subunits were detected with horse-SARS antibody (lane 3). The full-length S protein was also detected by all of the three antibodies used (lanes 1, 2 and 3). The sizes of S, S1 and S2 are the same as those mature forms detected in the media of infected cells. The results clearly showed that the cleavage products S1 and S2 as well as the full-length S were assembled into virions.

It was observed that 62/64-kDa proteins, also detected in transfected cells (see FIG. 1b), were identified in the lysate of infected cells (FIG. 2a, lane 2) but not in the cultured media (lane 4) and purified virions (FIG. 2b, lane 1 and 3). The 140-, 55- and 38 kDa proteins detected in transfected cells (shown in FIG. 1b) were not found in infected cells and purified viral particles, suggesting that they were not assembled into virion. The 140-kDa protein might be the unglycosylated form of S. It seemed that the 140-, 55- and 38-kDa proteins were produced only in S-transfected cells.

Detection of Cleavage Products of S in Transfected Cells—As no typical motif (RRAR/HR) immediately upstream of the cleavage site for coronaviruses was found in S of SARS CoV, it was necessary to determine experimentally the sequence harboring the alternative cleavage site between S1 and S2 subunits. For this purpose, six C-terminally-truncated S constructs (S 11 to S 16, see FIG. 1a) were expressed in Cos-7 cells. If the S protein was cleaved, the C-terminal products would become smaller when mutants with the C-terminal deletions were used. On the other hand, the size of the N-terminal products would remain the same when these mutants were used. Additional polyclonal and monoclonal antibodies, raised against different regions and domains, were used to detect all potential cleavage and/or other S-derived products. Rabbit-S 2, rabbit-S 3 and rabbit-S 9 recognized regions within residues 362-790 (S 2), 168-461 (S 3) and 798-1055 (S 9) (see FIG. 1a). Monoclonal antibodies MAb12-6 and MAbC1 recognized domains containing residues 281-300 and 631-650 of S, respectively.

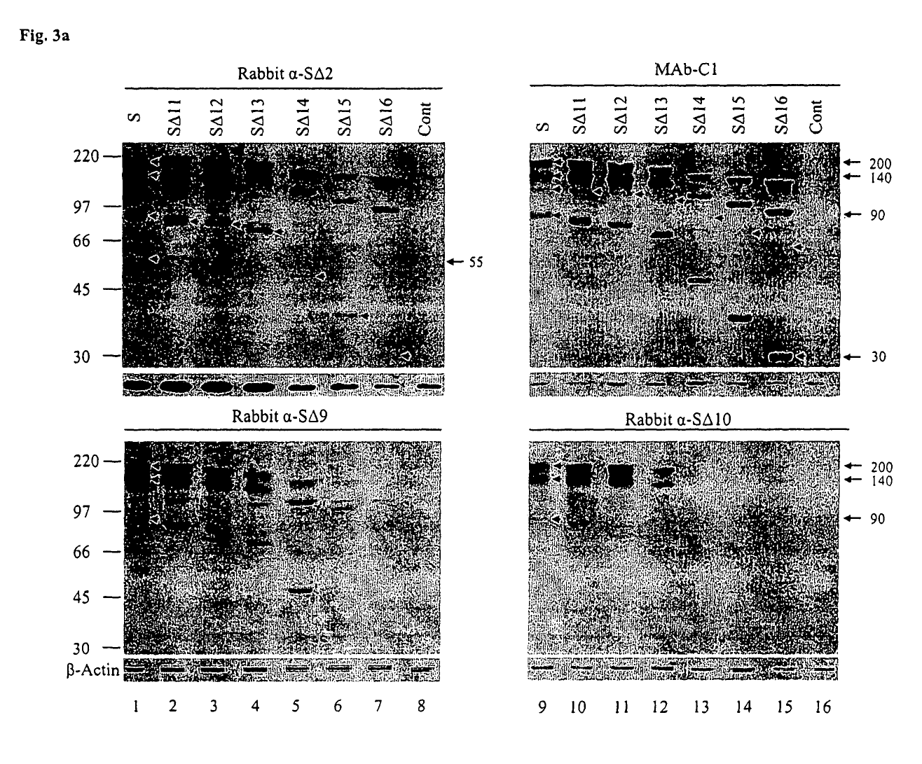

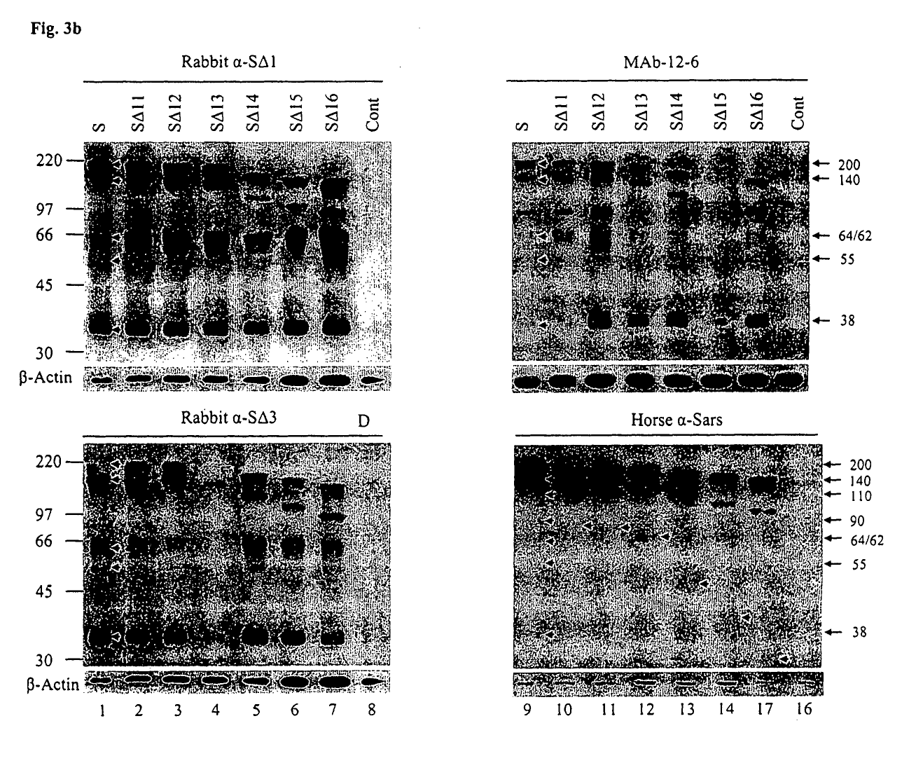

(1) Detection of the C-terminal Products—Western blot analysis was carried out to detect the S proteins in Cos-7 cells transfected with the full-length S and S 11 to S 16 constructs. As shown in FIG. 3a, 200-, 140- and 90-kDa bands were detected in cells expressing the full-length S protein when probed with rabbit-S 2, -S 9, -S 10 and MAbC1 antibodies (lanes 1 and 9, upper and low panels). Products progressively smaller than the 200- and 140-kDa bands were detected in cells expressing the C-terminal deletion mutants (S 11 to 16) with the same four antibodies (FIG. 3a, lanes 2 to 7 and 10 to 15). Among them, rabbit-S 2 and monoclonal MAbC1 were able to detect the corresponding products from each of the deletion constructs (FIG. 3a, upper panels, lane 2 to 7 and 10 to 15). Rabbit-S 9 and rabbit-S 10 only recognized the products derived from larger truncated-mutants as expected (FIG. 3a, lower panels) (also see FIG. 1a). Their apparent molecular masses are consistent with their calculated molecular weights and their predicted molecular weight changes after glycosylation, suggesting that they are the full length glycosylated and unglycosylated forms, respectively, of the products encoded by each constructs.

Similarly, products progressively smaller than the 90-kDa protein were detected from the C-terminal deletion constructs (S 11 to 16) by the same C-terminus-specific antibodies (FIG. 3a, lanes 1 to 7 and 9-15, as indicated by arrows in lanes 1 to 7 in upper panel). The migration patterns of these products and their apparent molecular masses strongly suggest that they represent the C-terminal cleavage products of the S protein. The S 16 protein contains the N-terminal 797 amino acid residues and its C-terminal cleavage product is 30-kDa in size (lanes 7 and 15, upper panels). When monoclonal antibody MAbC1 was used, the 30-kDa product derived from S 16 was detected (lane 15, upper panel), indicating that the cleavage site was present upstream of the binding sequence of this antibody (residues 631-650).

It was noted that a 55-kDa protein (lanes 1 to 7, upper panel) was detected by rabbit-S 2 instead of a large S1 product. The 55-kDa protein was not detected by monoclonal antibody MAbC1 (lanes 9 to 15, upper panel). It was also noted that another set of progressively smaller, relatively-faint products were detected when rabbit-S 2 and MAbC1 was used (lanes 1 to 7 and 9 to 15, upper panels, as indicated by arrows in lanes 9 to 15 in upper panel). They might be the intermediate products, suggesting that the S1 might be degraded or cleaved in transfected cells under conditions used in this study. This explained why a 110-kDa S1 was not detected with rabbit-S 2, which covers the residues from 362 to 790 and has the potential to recognize both S1 and S2.

(2) Detection of the N-terminal Products—Rabbit-S 1, rabbit-S 3 and monoclonal antibody MAb12-6 were used to detect the N-terminal products of S in cells transfected with the same constructs described above. For the full-length construct (FIG. 3b, lanes 1 and 9), the 200-, 140-, 64-/62-, 55- and 38-kDa bands were observed when the three antibodies were used (indicated by arrows). It was observed that the 140-kDa protein (as well as 200-kDa protein) was also detected by these N-terminally-specific antibodies, confirming that it was the full-length unglycosylated form of S. For the C-terminally-truncated S constructs S 11 to 16, the two bands at the top in each lane were the full-length proteins, glycosylated and unglycosylated, encoded by each truncated constructs (FIG. 3b, lanes 2 to 7 and 9 to 15). The 64-/62-, 55- and 38-kDa proteins were detected in cells expressing all constructs by these N-terminally specific antibodies (FIG. 3b, lanes 1 to 7 and 9 to 15). The sizes of the 64-/62-, 55- and 38-kDa proteins remained the same when the C-terminally-truncated mutants were used, confirming that they were the N-terminal products of S.

The horse-α-SARS antibody was used to detect proteins in the same cell lysates. This antibody can detect both the N- and C-terminal products. All the N- and C-terminal proteins described above were detected (FIG. 3b, lanes 9 to 15, lower panel), confirming that all the described proteins were specifically S-derived.

The 110-kDa S1 was not detected (or detected as a weak band with horse-SARS, see FIG. 1b) in transfected cells under conditions used. Instead, smaller N-terminal products were detected. The results strongly suggested that the S1 was degraded rapidly in this expression system. This might also happen in virus-infected cells, as 64/62-kDa proteins were also detected in infected cells (see, FIG. 2a, lane 2). Nevertheless, this expression and processing system makes it possible to map the cleavage site sequences for the SARS CoV S protein.

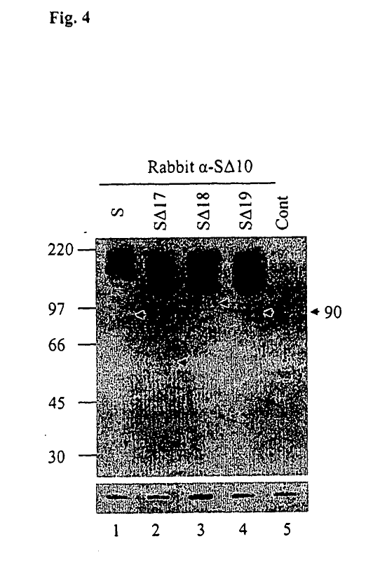

Mapping of Amino Acid Sequence Harboring the Cleavage Site—For the full-length S protein, the C-terminal cleavage product was 90-kDa in size. For the shortest truncated construct S 16 (encoding residues 1 to 797), the cleaved product was 30-kDa in size. The monoclonal antibody MAbC1 recognized the residues from 631 to 650. Therefore, the putative cleavage site of S1 and S2 might be located in the region around amino acid residue 600. To determine the sequence involved in the cleavage of S, several internally-deleted mutants of the S gene were constructed. If deleted sequence contains the cleavage site, the 90-kDa would not be produced. First, three mutants were expressed, which carried deletions from residues 601-800 (S 17), 401-600 (S 18) and 201-400 (S 19). The S specific proteins were analyzed by Western blot using rabbit-S 10. As seen in FIG. 4, in cells transfected with S 17, a protein, 200-residues shorter than 90-kDa, was observed (lane 2), indicating that the cleavage site might be upstream of residue 600. The deletion of residues 401-600 (S 18) indeed abolished the 90-kDa cleavage product (lane 3). When residues 201-400 (S 18) were deleted, the 90-kDa product was still produced as expected (lane 4). In fact, a slightly larger product than the 90-kDa S2 was detected in cells transfected with S 18, providing further evidences that S1 was probably cleaved in the middle of S1 by an endoprotease. This was consistent with observations that antibodies against the C-terminus detected a set of intermediate cleavage products (see FIG. 3a, indicated by arrows in lanes 8 to 15, upper panel).

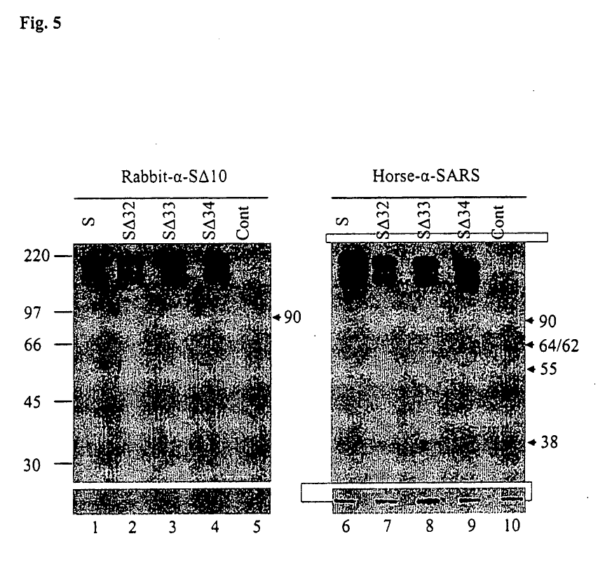

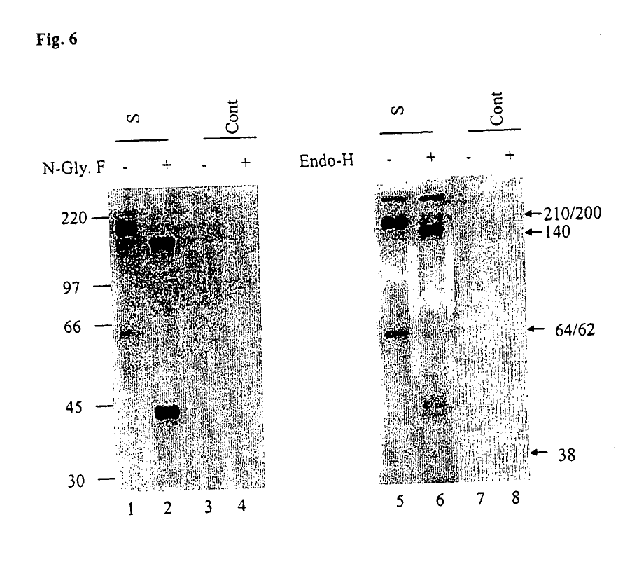

To further define the sequence harboring the cleavage site, three more deletion mutants were expressed and analyzed. These constructs encoded the S proteins with deletions from residues 531-550 (S 32), 551-570 (S 33) and 571-590 (S 34). Western blot analysis using rabbit-S 10 (FIG. 4b) showed that, when residues 551-570 (S 33) were removed, the production of the 90-kDa protein was abolished (lane 3). The deletions of 531-550 (S 32) and 571-590 (S 34) did not affect the detection of the 90-kDa cleavage product (lanes 2 and 4). This was confirmed when horse-SARS was used to analyze the same cell lysates (FIG. 5, lanes 6 to 10). It was noted that both the 90-kDa and the 55-kDa protein was not detected only when S 33 was expressed, indicating that the 55-kDa protein was the cleavage or degraded product of S1. The sequence of the 20 residues deleted in S 33 was FGRDVSDFTDSVRDPKTSEI. A BLAST search against amino acid sequences of the SARS CoV S showed that the residues 551 to 570 were fully conserved among different isolates published to date (data not shown). Unlike the S protein of other coronaviruses, no RRARR or RRAHR (or even paired basic residues) motif existed within this small region. The results strongly suggested that the cleavage site of the SARS CoV S protein was different from those of other coronaviruses and that other cellular proteases rather than the furin-like ones were involved.

Further Investigation of Glycosylation of the S Protein by Endoglycosidase-H (Endo-H) and N-glycosidase F (N-gly-F) Treatment—As shown in FIG. 6, the S proteins in transfected cells were treated with N-gly-F (lanes 1 to 4) and Endo-H (lanes 5 to 8). The results showed that the 200-kDa protein was a glycosylated form of S as it was sensitive to the treatment with these two enzymes. The 140-kDa protein was an unglycosylated, full-length S as the treatment of these enzymes had no effects on it (FIG. 6, lanes 1, 2, 5 and 6). It was interesting to find that a top band, slightly larger than 200-kDa protein, was an Endo-H resistant (lanes 5 and 6). The results confirmed that the 200-kDa protein was further modified as shown in infected cell culture (described earlier in this study), perhaps before incorporated into virions. The results also proved that 64/62- and 38-kDa proteins were also the glycosylated forms of the N-terminal products. It would be interesting to examine if the 90- and 55-kDa proteins were glycosylated forms or Endo-H resistant forms. However, as the efficiency of cleavage was much lower in transfected cells, they were not detectable under the conditions used in this study.

Generation of rabbit antibodies against the different regions on S protein. Previous studies using mammalian-expressed native S constructs have found that antibodies targeting 270-510, 548-567 and 607-627 of S in S1 region to be neutralizing (11, 30), and 803-828 in the S2 region were also found to possess neutralizing properties (13). However, the lack of post-translational modification such as glycosylation in a bacterial expression system might exhibit different potential sites for eliciting neutralizing antibodies. To study the neutralizing region on bacterial-expressed SARS CoV S protein, we cloned 5 DNA constructs of S covering the entire ectodomain (FIG. 1). We cloned 2 fragments covering the S1 region (S 1, S 3), 2 fragments covering the S2 region (S 9, S 10) and 1 fragment covering a portion of both S1 and S2 (S 2). The constructs S 9 and S 10 contain the heptad repeat 1 (HR1) and heptad repeat 2 (HR2) regions respectively. Antigens for constructs S 1, S 2, S 3, S 9 and S 10, were produced using a bacterial-expression system. These antigens were injected into rabbits to raise polyclonal antibodies against their respective target regions. Two rabbits were used to raise antibodies against each respective antigen. Two weeks after the initial immunization, the rabbits were given booster injections at three-week intervals. 10 ml of blood were harvested from the rabbits each time after the 4^{th}, 6^{th}, 8^{th}, 12^{th}, 14^{th} and 16^{th} injections. The polyclonal antibodies were characterized with Western Blot analysis, immunoprecipitation and immunofluorescence.

Specificity of rabbit antibodies to the SARS CoV S protein in Western Blot. The specificity of the rabbit antibodies for the full length S protein expressed in mammalian cells are determined by Western Blot analysis. As postulated, anti-S antibodies from the serum of a patient (P6) who has recovered from SARS-CoV infection (26), could detect 2 major bands of full-length S protein, the 140 kDa unglycoslyated form and 200 kDa glycoslyated form (FIG. 2a), in the lysates of Cos7 cells transfected with pKT-S. These are specific S bands as they were not detected in the negative control. We observed similar results using the antibodies that we have raised against the 5 S recombinant proteins (FIG. 2b-f). This indicates that all the antibodies raised against the various S constructs could specifically bind to S in denaturing condition by targeting different regions of the linearized S protein. Therefore, these antibodies are specific to the denatured full-length SARS-CoV S protein.

Detection of the native form of SARS CoV in immuoprecipitation. To determine the specificity of the antibodies for the native S protein and their respective target regions on the S protein, we carried out immunoprecipitation experiments using the various antibodies. Lysates of Cos7 cells infected with T7 vaccinia virus and transfected with pKT-S and C-terminal deletion mutants, pKT-S 11, pKT-S 12, PKT-S 13, pKT-S 14, pKT-S 15 and pKT-S 16, were immunoprecipitated with P6 serum and the 5 antibodies raised against S 1, S 2, S 3, S 9 and S 10. The SARS CoV S protein and the C-terminal deletion recombinant proteins can be detected by immunoprecipitation with the P6 serum (FIG. 3f). The core-glycosylated S protein (200 kDa) and the fully glycosylated S protein (210 kDa) were also clearly detected when we use antibodies raised against the various recombinant S proteins (FIG. 3a-e, Lane 1). Antibodies targeting the region S 1, S 2 and S 3 respectively can detect the full-length S and all the C-deletion mutants (FIG. 3a-c). Antibodies targeting the region S 9 can detect full-length S and pKT-S 11 to pKT-S 15. No specific band was detected in the PKT-S 16 lane because the PKT-S 16 does not include the S 9 region (FIG. 3d). Antibodies targeting against S 10 only detect the protein bands expressed by PKT-S 11 to PKT-S 13 (FIG. 3e). The region expressed by pKT-S 14 to pKT-S 16 does not include the S 10 region and thus rabbit anti-S 10 does not detect any band in these lanes, showing that these antibodies are highly specific to their target region. The native conformation of the S protein was retained in lysis buffer in immunoprecipitation and the results showed that they possessed epitope(s) which can be recognized by all the 5 antibodies raised against the 5 recombinant proteins.

Maturation of the 200 kDa S protein to the 210 kDa EndoH resistant form. Two S specific bands were observed in the immunoprecipitation experiments. To find out the relationship between the 2 bands, we carried out a time-course experiment. Cos7 cells transfected with pKT-S, labeled with ³⁵S-met and immunoprecipitated with rabbit anti-S 10 were harvested at ½ h, 1 h, 2 h, 4 h and 6 h. The results showed a gradual increase in the 210 kDa band accompanied by a gradual decrease in the 200 kDa band (FIG. 4, Lane 1-8). An EndoH experiment is done to find out whether the bands were sensitive to EndoH treatment. Cos7 cells were subjected to T7 vaccinia virus infection, transfected with pKT-S and treated with EndoH enzyme whereas control cells were not treated with EndoH. Results showed that the 210 kDa band was EndoH-resistant and the 200 kDa band was EndoH-sensitive (FIG. 4, Lane 9, 10). Hence, the results demonstrated the maturation of the 200 kDa band to the 210 kDa band.

Detection of SARS CoV S protein on surface of Cos7 cells. To provide further evidence that the antibodies raised were able to recognize the native form of S protein, we performed immunofluorescence experiments. The Cos7 cells utilised for the indirect immunofluorescence were non-permeabilized. After infection with T7 vaccinia virus and transfection with PKT-S as described above, sufficient time (8 h post-transfection) was allowed for the S protein to be expressed and transported to the cell surface and these can be clearly detected using any of the antibodies raised against the various S protein constructs (FIG. 5). Green fluorescence indicated the position of the various primary antibodies that bind to the Cos7 cells. S protein was expressed and processed in their native conformation by the Cos7 cells. The binding of the antibodies against S 1, S 2, S 3, S 9 and S 10, to the S protein on the cell surface provide further evidence that these antibodies are specific and sensitive to the native conformation of SARS CoV S protein.

A region in S2 can elicit neutralizing activity. All serum from rabbits injected with the various S proteins were tested for neutralizing activity after each bleed. Results for rabbits injected with PGEX-S 1, S 2, S 3 and S 9 showed negative response. Serum from rabbits injected with pGEX-S 10 showed neutralizing activities after the 4^{th} injection. Initial tests using SARS-CoV at 200 TCID₅₀ showed high titers (1:364) of neutralizing antibodies in all the rabbit anti-S 10 bleeds, beginning with serum bled after the 8^{th} injection (Table 3). The 16^{th} injection is the last booster injection and the rabbit is sacrificed at this stage. To ascertain the results above, serum samples after the 4^{th}, 6^{th}, 8^{th}, 12^{th}, 14^{th} and 16^{th} injection with pGEX-S 10 was sent for a neutralizing test using SARS-CoV at 1000 TCID₅₀ (Table 4). Neutralizing activities was found to be as high as 1:189.2 at 1000 TCID₅₀, and is comparable, if not, higher than the titers detected in SARS patients. Antibody response in SARS patients at 100 TCID₅₀ ranges from 1:150 to 1:475 over a period of 210 days (35). This result provides strong evidence that S 10 is the region that can stimulate the production of neutralizing antibodies against the SARS CoV.

TABLE 3
Neutralizing test at 200 TCID₅₀

| No. of injections | Neutralizing titers |
|---|---|
| before bleeding | (Average of 2 readings) |
| 0 (Pre-immune | 0 |
| serum) | |
| 8 | 1:70 |
| 12 | 1:364 |
| 14 | 1:208 |
| 16 | 1:256 |
| Control (water) | 0 |

TABLE 4
Neutralizing test at 1000 TCID₅₀

| No. of injections | Neutralizing titers |
|---|---|
| before bleeding | (Average of 2 readings) |
| 0 (Pre-immune | 0 |
| serum) | |
| 4 | 1:30 |
| 6 | 1:66.8 |
| 8 | 1:112 |
| 12 | 1:67.2 |
| 14 | 1:98 |
| 16 | 1:189.2 |
| Control (water) | 0 |

SEQ ID NO. 1—The full-length nucleotide sequence of the spike (S) gene of SARS CoV, clone 12 of 2774 stain. RNA linear from nucleic acid 1 to 3765.

ORGANISM: SARS coronavirus 2774 strain Viruses; ssRNA positive-strand viruses, no DNA stage; Nidovirales; Coronaviridae; Coronavirus.

SEQ ID NO. 2—The full-length amino acid sequence of the spike (S) gene of SARS CoV, clone 12 of 2774 strain. Amino acid 1-1255.

SEQ ID NO. 3—The nucleotide sequence of SΔ10 fragment of the spike (S) gene of SARS CoV, clone 12 of 2774 strain. Nucleic acid 3087-3581.

SEQ ID NO. 4—The amino acid sequence of SΔ10 fragment of the spike (S) gene of SARS CoV, clone 12 of 2774 strain. Amino acid 1029-1192.

SEQ ID NO. 5—The amino acid sequence of the neutralizing fragment of the spike (S) gene of SARS CoV, clone 12 of 2774 strain. Amino acid 1055-1192.

SEQ ID NO. 6—The amino acid sequence of SΔ11 fragment of the spike (S) gene of SARS CoV, clone 12 of 2774 strain. Amino acid 1-1232.

SEQ ID NO. 7—The amino acid sequence of SΔ17 fragment of the spike (S) gene of SARS Cov, clone 12 of 2774 strain. Amino acid 1-1255 with a deletion from 601 to 800.

SEQ ID NO. 8—The amino acid sequence of SΔ18 fragment of the spike (S) gene of SARS CoV, clone 12 of 2774 strain. Amino acid 1-1255 with a deletion from 401 to 600.

SEQ ID NO. 9—The amino acid sequence of SΔ19 fragment of the spike (S) gene of SARS CoV, clone 12 of 2774 strain. Amino acid 1-1255 with a deletion from 201 to 400.

SEQ ID NO. 10—The amino acid sequence of SΔ20 fragment of the spike (S) gene of SARS CoV, clone 12 of 2774 strain. Amino acid 1-1255 with a deletion from 30 to 200.

All references cited herein, including U.S. Provisional Patent Application 60/528,596, filed Dec. 10, 2003, are incorporated by reference in their entirety.

### REFERENCES

1. Bosch B J, van der Zee R, de Haan C A and Rottier P J. 2003. The coronavirus spike protein is a class I virus fusion protein: structural and functional characterization of the fusion core complex. J. Virol. 77(16):8801-11.
2. Drosten, C., S. Gunther, W. Preiser, S. van der Werf, H. R. Brodt, S. Becker, H. Rabenau, M. Panning, L. Kolesnikova, R. A. Fouchier, A. Berger, A. M. Burguiere, J. Cinatl, M. Eickmann, N. Escriou, K. Grywna, S. Kramme, J. C. Manuguerra, S. Muller, V. Rickerts, M. Sturmer, S. Vieth, H. D. Klenk, A. D. Osterhaus, H. Schmitz, and H. W. Doerr. 2003. Identification of a novel coronavirus in patients with severe acute respiratory syndrome. N. Engl. J. Med. 348:1967-1976.
3. Fouchier, R. A., T. Kuiken, M. Schutten, G. van Amerongen, G. J. van Doornum, B. G. van den Hoogen, M. Peiris, W. Lim, K. Stohr, and A. D. Osterhaus. 2003. Aetiology: Koch's postulates fulfilled for SARS virus. Nature. 423:240.
4. Gallagher T M and Buchmeier M J. 2001. Coronavirus spike proteins in viral entry and pathogenesis. Virology. 279(2):371-4.
5. Gao W, Tamin A, Soloff A, D'Aiuto L, Nwanegbo E, Robbins P D, Bellini W J, Barratt-Boyes S and Gambotto A. 2003. Effects of a SARS-associated coronavirus vaccine in monkeys. Lancet. 362(9399):1895-6.
6. Koolen, M. J., M. A. J. Borst, M. C. Horzinek, and W. J. M. Spaan. 1990. Immunogenic peptide comprising a mouse hepatitis virus A59 B-cell epitope and an influenza virus T-cell epitope protects against lethal infection. J. Virol. 64:6270-6273.
7. Ksiazek, T. G., D. Erdman, C. S. Goldsmith, S. R. Zaki, T. Peret, S. Emery, S. Tong, C. Urbani, J. A. Corner, W. Lim, P. E. Rollin, S. F. Dowell, A. E. Ling, C. D. Humphrey, W. J. Shieh, J. Guarner, C. D. Paddock, P. Rota, B. Fields, J. DeRisi, J. Y. Yang, N. Cox, J. M. Hughes, J. W. LeDuc, W. J. Bellini, and L. J. Anderson; SARS Working Group. 2003. A novel coronavirus associated with severe acute respiratory syndrome. N. Engl. J. Med. 348:1953-1966.
8. Lai, M. M. C., and K. V. Holmes. 2001. Coronaviruses, p. 1163-1185. In D. M. Knipe, and P. M. Howley (ed.), Fields Virology, 4th ed. Lippincott, Philadelphia, USA.
9. Li W, Moore M J, Vasilieva N, Sui J, Wong S K, Berne M A, Somasundaran M, Sullivan J L, Luzuriaga K, Greenough T C, Choe H and Farzan M. 2003. Angiotensin-converting enzyme 2 is a functional receptor for the SARS coronavirus. Nature. 426(6965):450-4.
10. Olsen and C W. 1993. A review of feline infectious peritonitis virus: molecular biology, immunopathogenesis, clinical aspects, and vaccination. Vet Microbiol. 36(1-2):1-37.
11. Parker, M. D., D. Yoo, G. J. Cox, and L. A. Babiuk. 1990. Primary structure of the S peplomer gene of bovine coronavirus and surface expression in insect cells. J. Gen. Virol. 71:263-270.
12. Peiris, J. S., S. T. Lai, L. L. Poon, Y. Guan, L. Y. Yam, W. Lim, J. Nicholls, W. K. Yee, W. W. Yan, M. T. Cheung, V. C. Cheng, K. H. Chan, D. N. Tsang, R. W. Yung, T. K. Ng, and K. Y. Yuen; SARS study group. 2003. Coronavirus as a possible cause of severe acute respiratory syndrome. Lancet 361:1319-1325.
13. Siddell and S. G. 1995. The Coronaviridae, Plenum Press, New York, USA.
14. Spaan, W. J. M., D. Cavanagh, and M. C. Horzinek. 1988. Coronaviruses: structure and genome expression. J. Gen. Virol. 69:2939-2952.
15. Vennema, H., L. Heijnen, A. Zijderveld, M. C. Horzinek, and W. J. Spaan. 1990. Intracellular transport of recombinant coronavirus spike proteins: implications for virus assembly. J. Virol. 64:339-346.
16. Xiao X, Chakraborti S, Dimitrov A S, Gramatikoff K and Dimitrov D S. 2003. The SARS-CoV S glycoprotein: expression and functional characterization. Biochem Biophys. Res Commun. 312(4):1159-64.
39. Ksiazek, T. G., D. Erdman, C. S. Goldsmith, S. R. Zaki, T. Peret, S. Emery, S. Tong, C. Urbani, J. A. Corner, W. Lim, P. E. Rollin, S. F. Dowell, A. E. Ling, C. D. Humphrey, W. J. Shieh, J. Guarner, C. D. Paddock, P. Rota, B. Fields, J. DeRisi, Y. J. Yang, N. Cox, J. M. Hughes, J. W. LeDuc, W. J. Bellini, L. J. Anderson; SARS working group. 2003. A novel coronavirus associated with severe acute respiratory syndrome. N. Engl J. Med. 348:1953-66
40. Marra, M. A., S. J. Jones, C. R. Astell, R. A. Holt, A. Brooks-Wilson, Y. S. Butterfield, J. Khattra, J. K. Asano, S. A. Barber, S. Y. Chan, A. Cloutier, S. M. Coughlin, D. Freeman, N. Girn, O. L. Griffith, S. R. Leach, N. Mayo, H. McDonald, S. B. Montgomery, P. K. Pandoh, A. S. Petrescu, A. G. Robertson, J. E. Schein, A. Siddiqui, D. E. Smailus, J. M. Stott, G. S. Yang, F. Plummer, A. Andonov, H. Artsob, N. Bastien, K. Bernard, T. F. Booth, D. Bowness, M. Czub, M. Drebot, L. Fernando, R. Flick, M. Garbutt, M. Gray, A. Grolla, S. Jones, H. Feldmann, A. Meyers, A. Kabani, Y. Li, S. Normand, U. Stroher, G. A. Tipples, S. Tyler, R. Vogrig, D. Ward, B. Watson, R. C. Brunham, M. Krajden, M. Petric, D. M. Skowronski, C. Upton, R. L. Roper. 2003. The genome sequence of the SARS-associated coronavirus. Science. 300: 1399-404.
41. Rota, P. A., M. S. Oberste, S. S. Monroe, W. A. Nix, R. Campagnoli, J. P. Icenogle, S. Penaranda, B. Bankamp, K. Maher, M. H. Chen, S. Tong, A. Tamin, L. Lowe, M. Frace, J. L. DeRisi, Q. Chen, D. Wang, D. D. Erdman, T. C. Peret, C. Burns, T. G. Ksiazek, P. R. Rollin, A. Sanchez, S. Liffick, B. Holloway, J. Limor, K. McCaustland, M. Olsen-Rasmussen, R. Fouchier, S. Gunther, A. D. Osterhaus, C. Drosten, M. A. Pallansch, L. J. Anderson, W. J. Bellini. 2003. Characterisation of a novel coronavirus associated with severe acute respiratory syndrome. Science. 300: 1394-9.
42. Sturman, L. S., Ricard, C. S., and Holmes, K. V. (1985) J. Virol. 56, 904-911
43. Yamada, Y. K., Takimoto, K., Yabe, M., and Taguchi, F. 1997. Virology 227, 215-219
44. Bos, E. C., Luytjes, W., and Spaan, W. J. (1997) J. Virol. 71, 9427-9433
45. Hingley, S. T., Gombold, J. L., Lavi, E., and Weiss, S. R. (1994) Virology 200, 1-10
46. Bos, E. C., Heijnen, L., Luytjes, W., and Spaan, W. J. (1995) Virology 214, 453-463
47 Gotoh, B., Ohnishi, Y., Inocencio, N. M., Esaki, E., Nakayama, K., Barr, P. J., Thomas, G., and Nagai, Y. (1992) J. Virol. 66, 6391-6397
48. Gotoh, B., Ogasawara, T., Toyoda, T., Inocencio, N. M., Hamaguchi, M., and Nagai Y. (1990) EMBO J. 9, 4189-4195
49. Luytjes, W., Gerritsma, H., Bos, E., and Spaan. W. (1997) J. Virol. 71, 949-955
50. Niemann, H., and Klenk, H. D. (1981) J. Mol. Biol. 153, 993-1010
51. Rottier, P. J. M., Horzinek, M. C., and Van der Zeijst, B. A. M. (1981) J. Virol. 40, 350-357
52. Simmons, G., Reeves, J. D., Rennekamp, A. J., Amberg, S. M., Piefer, A. J., and Bates, P. PNAS 101, 4240-4245
53. Shen, S., Wen, Z. L., and Liu D. X. (2003) Virology 311, 16-17

1. A peptide or protein fragment of SEQ ID NO.1, said fragment comprising a neutralizing domain.

2. The peptide or protein of claim 1 which comprises the sequence of amino acid selected from the group SEQ ID NO. 4, SEQ ID NO. 5, SEQ ID NO. 6, SEQ ID NO. 7, SEQ ID NO. 8, SEQ ID NO. 9 or SEQ ID NO. 10.

3. The peptide or protein of claim 1 having the HR2 heptad region of the coronavirus S protein.

4. The peptide or protein of claim 1 selected from the group consisting essentially of SEQ ID NO. 4, SEQ ID NO. 5, SEQ ID NO. 6, SEQ ID NO. 7, SEQ ID NO. 8, SEQ ID NO. 9 or SEQ ID NO. 10.

5. The peptide or protein of claim 1 wherein the fragment comprises SEQ ID NO.4.

6. The peptide or protein of claim 1 wherein the fragment comprises SEQ ID NO.5.

7. A method of producing a fragment of the S protein of coronavirus comprising the steps of:
a) transforming a cell with a nucleic acid encoding a fragment of the S protein of coronavirus the fragment comprising a neutralizing domain, said nucleic acid in operative association with regulatory sequences capable of directing the expression thereof in the cell;
b) expressing the protein fragment in the cell; and
c) isolating the protein fragment.

8. An antibody to a peptide or protein fragment of SEQ ID NO.1, said fragment comprising SEQ ID NO. 5.

9. The antibody of claim 8 wherein the fragment comprises SEQ ID NO. 4.

10. The antibody of claim 8 wherein the peptide or protein comprises the HR2 heptad region of the coronavirus S protein.

11. The antibody of claim 8 wherein the peptide or protein is selected from the group consisting of SEQ ID NO. 4, SEQ ID NO. 5, SEQ ID NO. 6, SEQ ID NO. 7, SEQ ID NO. 8, SEQ ID NO. 9 or SEQ ID NO. 10.

12. A method of detecting a SARS coronaviral infection in a patient comprising the step of applying the antibody of claim 8 to at least part of the cells collected from the patient.

13. A kit for the detection of SARS coronavirus containing the antibody of claim 8.

14. A method to treat a patient with severe acquired respiratory syndrome or prevent the onset thereof comprising administering to the patient the peptide or protein of claim 1.

15. A method to treat a patient with severe acquired respiratory syndrome or prevent the onset thereof comprising administering to the patient the antibody of claim 8.

16. A vaccine to treat a patient with severe acquired respiratory syndrome or prevent the onset thereof comprising an effective amount of the peptide or protein of claim 1.

17. A vaccine to treat a patient with severe acquired respiratory syndrome or prevent the onset thereof comprising an effective amount of the antibody of claim 8.

18. An antibody to an S protein fragment selected from the group consisting of S 1, S 2, S 3, and S 9.

19. A method of detecting a SARS coronaviral infection in a patient comprising the step of applying the antibody of claim 18 to at least part of the cells collected from the patient.

20. A kit for the detection of SARS coronavirus containing the antibody of claim 18.

21. A mature, glycosylated spike protein of a coronavirus.

22. The mature glycosylated spike protein of claim 21 wherein the mature glycosylated spike protein contains a transmembrane domain (TMD).

23. The mature glycosylated spike protein of claim 21 wherein the mature glycosylated spike protein is a 210 KDa protein.

24. The mature glycosylated spike protein of claim 21 wherein the coronavirus is a SARS coronavirus.

25. The mature glycosylated spike protein of claim 21 wherein the coronavirus is a SARS coronavirus strain, 2774.

26. A method of producing a mature, glycosylated spike protein of a coronavirus comprising the steps of
a) transforming a cell with a nucleic acid encoding a spike protein of a coronavirus or part there of;
b) expressing the spike protein in the cell; and
c) isolating the mature, glycosylated spike protein of the coronavirus.

27. The method of claim 26 wherein the mature glycosylated spike protein contains a transmembrane domain (TMD).

28. The method of claim 26 wherein the mature glycosylated spike protein is a 210 KDa protein.

29. The method of claim 26 wherein the coronavirus is a SARS coronavirus.

30. The method of claim 26 wherein the coronavirus is a SARS coronavirus strain, 2774.

31. The method of claim 26 wherein the cell is selected from the group consisting of human lung cell and cos-7 cell.

32. A method of screening for a mature, glycosylated spike protein of a coronavirus comprising the steps of
a) isolating a spike protein;
b) immunoprecipitating the isolated spike proteins with Endo-H; and
c) detecting the remaining spike proteins that are the mature glycosylated spike protein.

33. The method of claim 32 wherein the mature glycosylated spike protein contains a transmembrane domain (TMD).

34. The method of claim 32 wherein the mature glycosylated spike protein is a 210 KDa protein.

35. The method of claim 32 wherein the coronavirus is a SARS coronavirus.

36. The method of claim 32 wherein the coronavirus is a SARS coronavirus strain, 2774.

37. An antibody to a mature, glycosylated spike protein of a coronavirus or part their of.

38. The antibody of claim 37 wherein the mature glycosylated spike protein contains an anti-transmembrane domain (TMD).

39. The antibody of claim 37 wherein the mature glycosylated spike protein is a 210 KDa protein.

40. The antibody of claim 37 wherein the coronavirus is a SARS coronavirus.

41. The antibody of claim 37 wherein the coronavirus is a SARS coronavirus strain, 2774.

42. A method of detecting a SARS coronaviral infection in a patient comprising the step of applying the antibody of claim 37 to at least part of the cells collected from the patient.

43. A kit for the detection of SARS coronavirus containing the antibody of claim 37.

44. A method to treat a patient with severe acquired respiratory syndrome or slow the progression of severe acquired respiratory syndrome comprising administering to a patient in need thereof the antibody of claim 37.

45. A vaccine to treat a patient with severe acquired respiratory syndrome or prevent the onset of severe acquired respiratory syndrome comprising the mature glycosylated spike protein of claim 21.

46. A vaccine to treat a patient with severe acquired respiratory syndrome or prevent the onset of severe acquired respiratory syndrome comprising the antibody of claim 37.

47. A vaccine to treat a patient with severe acquired respiratory syndrome or prevent the onset of severe acquired respiratory syndrome comprising the mature glycosylated spike protein of claim 22.

48. A vaccine to treat a patient with severe acquired respiratory syndrome or prevent the onset of severe acquired respiratory syndrome comprising the antibody of claim 38.
